(19) United States
(12) Patent Application Publication
(10) Pub. No.: US 2026/0216284 A1
(43) Pub. Date: Jul. 30, 2026

(54) STABILIZED ENGRAILED PROTEIN AQUEOUS COMPOSITIONS

(71) Applicant: BRAINEVER, Paris (FR)

(72) Inventor: Hubert MECHAIN, Illkirch-Graffenstaden (FR)

(21) Appl. No.: 19/146,627

(22) PCT Filed: Jan. 10, 2024
(86) PCT No.: PCT/EP2024/050442

§ 371 Date: Jul. 9, 2025

(30) Foreign Application Priority Data
Jan. 13, 2023 (EP) 23305048.3

(51) Int. Cl. A61K 38/17; A61K 9/08; A61K 47/18; A61K 47/26

(52) U.S. Cl. CPC A61K 38/1709; A61K 9/08; A61K 47/183; A61K 47/26

(57) ABSTRACT

The invention relates to a formulation of Engrailed (EN) protein, more particularly an aqueous formulation of Engrailed (EN) protein, that is capable to inhibit or minimize EN protein aggregation. In some embodiments, the formulation of the invention is capable to inhibit or minimize EN protein dimerization and/or oligomerization, the EN protein being the monomer.

### TECHNICAL FIELD

The invention relates to special formulations for obtaining stable aqueous protein compositions. In particular, the invention relates to stabilized aqueous compositions of Engrailed (EN) protein comprising specific combinations of stabilizing compounds.

### Technological Background

Neurodegenerative diseases are a group of irreversible neurological diseases caused by the loss of neuronal cells in the brain and spinal cord, and mainly include Alzheimer's disease, Parkinson's disease, amyotrophic lateral sclerosis (ALS), Huntington's disease, etc. Most degenerative diseases of the nervous system still lack effective treatment methods, so finding effective methods for preventing, delaying and treating the diseases is a problem to be solved urgently.

Amyotrophic lateral sclerosis (ALS) is a progressive nervous system disease that affects nerve cells in the brain and spinal cord, causing loss of muscle control. It is either sporadic or familial origin and causing the death of motor neurons. ALS starts with initial symptoms such as weakness in hands, movement disorders with fingers and fascicular contraction in upper limbs. Thereafter, ALS causes amyotrophia and/or muscular weakness, bulbar paralysis and fascicular contraction in muscles, and it finally leads to respiratory failure and death within 3-5 years after disease onset.

Proposed treatments do not permit to reverse the damage of amyotrophic lateral sclerosis, but only to slow the progression of symptoms, prevent complications, and make people in need thereof more comfortable and independent.

To date, the antiglutamatergic small-molecule riluzole, antioxidant edaravone and, more recently, sodium phenylbutyrate and taurursodiol have been approved for the treatment of ALS with modest efficacy and dosing limited by important side effects such as hepatic toxicity and asthenia (Jaiswal M K., Med Res Rev, 2019; 39 (2), 733-748).

Therefore, there is still a need for new strategies capable of treating ALS, and more particularly of preventing and/or delaying the death of motor neurons.

One major issue in developing drug for treating central nervous diseases (CNS) diseases, such as ALS, is to guarantee a therapeutic level of the drug in the brain and spinal cord. On the pharmacokinetic side, it is thus required to enable the drug in question to pass the blood-brain barrier, which limits access to neuronal cells, and to reach motoneurons in a sufficient concentration, and over a reasonable time period. Thus the delivery route and formulation of the drug need to be fitted to that purpose. In ALS, deterioration of motor neurons occurs in the cortex, brainstem, and spinal cord, therefore direct intrathecal or intracerebroventricular administration of drug is preferred delivery route in patients with ALS.

Homeoproteins, or homeodomain proteins, are transcription factors that play a major role in cell migration and differentiation processes involved in morphogenesis. They are characterized by the presence of a sequence of 60 amino acids, the homeodomain, which is a DNA binding domain with a helix/turn/helix structure. It has been shown that the isolated domain of Antennapedia protein of Drosophila crosses the membrane of neurons in culture, accumulates in the nucleus and promotes neurite growth (EP0485578). The homeodomain is highly conserved and confers the internalization property to a large number of homeoproteins (Spatazza et al., 2013, Pharmacol. Rev. 65, 90-104).

Engrailed proteins (Engrailed-1 and Engrailed-2) are homeoproteins with similar biological activity that are designated collectively hereafter with the general term Engrailed (EN for the human or En1/2 for mice). In neonates and in adults, Engrailed is expressed in cerebellar granule cells and in mesencephalic dopaminergic (DA) nuclei, including the substantia nigra pars compacta (SNpc that degenerates in Parkinson's disease), and the Ventral Tegmental Area (VTA). En1/2 plays important roles in the development of the midbrain and, in the midbrain, of the mesencephalic dopaminergic (mDA) neurons (Joyner, 1996, Trends Genet 12, 15-20). En1/2 also plays redundant roles in the survival of adult mDA neurons that are located in the SNpc and the VTA in the ventral part of the mesencephalon (Alberi et al., 2004, Development 131, 3229-3236), and therefore it was proposed for preventing or treating the loss of DA neurons in Parkinson disease. In WO 2013/128239, it was reported that local administration of En1/2 by infusion in the midbrain increases DA synthesis by DA neurons and associated motor activity. Prochiantz et al. (2011, FEBS Letters, 278, 52 (Abstract, Brunet et al., Nature 438:94-98, 2005 and Alvarez-Fisher et al., Nature Neurosci 14:1260-1266, 2011) have reported that Engrailed is not only a transcription factor but also a translation regulator that enhances the translation of mitochondrial mRNAs transcribed in the nucleus and it was shown that Engrailed transduction up-regulates the translation of Ndufs1 and Ndufs3, two proteins of mitochondrial complex I and increases ATP synthesis (see also Alvarez-Fischer et al., 2011, Nature Neuroscience, 14, 1260-1266, Stettler et al. 2012). Alternatively, in WO 2007/099227, it was shown that systemic administration of En1/2 to mice induces an increase in DA turnover in the striatum, reflected by an increase in the production of the DA metabolite 3,4-dihydroxyphenylacetic acid (DOPAC) without modification of dopamine levels. US20210379144 describes that accumulation of DNA damage has been linked to the process of aging and to the onset of age-related diseases including neurodegenerative disorders, such as ataxias, Alzheimer's, amyotrophic lateral sclerosis, Huntington's and Parkinson's diseases (Canugovi et al., 2013, 12, 578-587; Madabhushi et al., 2014, Neuron 83, 266-282)”.

Vargas Abonce et al. 2020 (bioRxiv 734020, https://doi.org/10.1101/734020) have shown that EN protein injection promotes motoneuron survival and motor functions and may be used for therapies alleviating the consequences of α-motoneuron degeneration.

The present invention is based on the fact that EN protein aggregates in liquid formulations of purified EN protein. However, according to FDA's guidance, it is critical for manufacturers of therapeutic protein products to minimize protein aggregation to the extent possible in order to decrease the potential for, and the risk associated with, an immune response that can cause some problems for both patient safety and product efficacy.

The present invention thus provides, among other aspects, a formulation of Engrailed (EN) protein, more particularly an aqueous formulation of Engrailed (EN) protein, that inhibits or minimizes EN protein aggregation. In some embodiments, the formulation of the Invention inhibits or minimizes EN protein dimerization and/or oligomerization, the EN protein being the monomer.

Protein aggregation relates to the process by which protein molecules assemble into complexes composed of two or more proteins, with the individual proteins being the monomer. Protein aggregation is typically driven by forces and interactions such as van der Waals and hydrophobic attractions, hydrogen bonding, electrostatic attractions. These types of interactions that occur between amino acids within the protein resulting in its folding also exist between amino acids in neighboring proteins, leading to protein aggregation. “Protein aggregation”, “protein dimerization” or “protein oligomerization” are synonyms according to the present invention.

In some embodiments, the formulation of Engrailed (EN) protein of the Invention delays the formation of EN dimers, oligomers and/or aggregation when stored for extended periods of time.

Therefore, in a preferred embodiment, the Invention relates to stabilized aqueous composition of Engrailed (EN) protein.

Advantageously, a composition of Engrailed (EN) protein of the Invention may be stored for extended periods of time without EN protein losing its biological activity or becoming overly dimerized, oligomerized and/or aggregated. In certain embodiments, the composition of Engrailed (EN) protein of the Invention may be stored for at least 6 months at temperatures up to at least about −65° C.

Preferably the present invention relates to an injectable composition of Engrailed (EN) protein for non-systemic administration to a subject in need thereof.

In a preferred embodiment, the composition of Engrailed (EN) protein of the Invention is suitable for the treatment of diseases or conditions associated with death of neurons, preferably motor neurons. In an even more preferred embodiment, the composition of the Invention is suitable for intrathecal or intracerebroventricular administration to a subject in need thereof.

The present invention further relates to methods using the composition of Engrailed (EN) protein of the Invention for treating subjects with diseases or conditions associated with death of neurons, preferably death of motor neurons. More particularly, the present invention provides methods for treating subjects with Amyotrophic lateral sclerosis (ALS). The invention further relates to methods of preventing and/or delaying the death of motor neurons.

According to the Invention, the methods of treatment include the administration of the composition of Engrailed (EN) protein of the Invention.

The present invention also relates to the composition of Engrailed (EN) protein of the invention for use in the treatment of subjects with diseases or conditions associated with death of neurons, preferably death of motor neurons.

The present invention also relates to the use of the composition of Engrailed (EN) protein of the invention for the manufacture of a drug for the treatment of subjects with diseases or conditions associated with death of neurons, preferably death of motor neurons.

The present invention further relates to methods of increasing the stability and/or reducing the aggregation of EN protein in aqueous solution, said method comprising admixing EN protein with specific formulation, wherein said formulation increases the stability and/or reduces the aggregation of EN protein in aqueous solution; and wherein said method provides a stable aqueous composition of EN protein.

### BRIEF DESCRIPTION OF THE DRAWINGS

The present disclosure will be more readily appreciated by reference to the following detailed description when being considered in connection with the accompanying drawings in which:

FIG. 1: pH effect on EN1 Protein oligomerisation measured by non-reduced SDS-page electrophoresis with Coomassie blue staining Lane 20: ladder; Lane 21 EN1 in 5 mM citrate/phosphate 250 mM Dextrose, 2.13 mM MgCl₂ pH 4 after dialysis; lane 22 EN1 in 5 mM citrate/phosphate 250 mM Dextrose, 2.13 mM MgCl₂ pH 4 after small scale ultrafiltration; lane 23 EN1 in 5 mM citrate/phosphate 250 mM Dextrose, 2.13 mM MgCl₂ pH 5 after dialysis; lane 24 EN1 in 5 mM citrate/phosphate 250 mM Dextrose, 2.13 mM MgCl₂ pH 5 after small scale ultrafiltration; lane 25 EN1 in 5 mM citrate/phosphate 250 mM Dextrose, 2.13 mM MgCl₂ PH 5 after dialysis; lane 26 EN1 in 5 mM citrate/phosphate 250 mM Dextrose, 2.13 mM MgCl₂ pH 5 after small scale ultrafiltration.

FIG. 2: pH effect on EN1 Protein oligomerisation measured by SEC-HPLC

Curve 1: 5 mM Citrate/Phosphate 250 mM Dextrose, 2.13 mM MgCl₂ pH 6; curve 2: 5 mM Citrate/Phosphate 250 mM Dextrose, 2.13 mM MgCl₂ pH 4; curve 3: 5 mM Citrate/Phosphate 250 mM Dextrose, 2.13 mM MgCl₂ pH 5.

FIG. 3: Glutathione effect on EN1 Protein oligomerisation measured by SEC HPLC and high molecular weight (~300 kDa) detected at 280 nm.

Curve 1: 5 mM Citrate/Phosphate 250 mM Dextrose, 2.13 mM MgCl₂ PH 5; curve 2: 5 mM Citrate/Phosphate 250 mM Dextrose, 2.13 mM MgCl₂ pH 5, 50 μM glutathione.

FIG. 4: Glutathione dose effect on EN1 Protein oligomerisation measured by SEC HPLC and high molecular weight (HMW) detected at 280 nm.

Curve 1: 5 mM Citrate/Phosphate 250 mM Dextrose, 2.13 mM MgCl₂ pH 5, 50 μM Glutathione; Curve 2: 5 mM Citrate/Phosphate 250 mM Dextrose, 2.13 mM MgCl₂ pH 5, 1 μM glutathione; curve 3/5 mM Citrate/Phosphate 250 mM Dextrose, 2.13 mM MgCl₂ pH 5, 100 μM Glutathione;

FIG. 5: Glutathione dose effect on EN1 Protein oligomerisation measured by non-reduced SDS-page electrophoresis with Coomassie blue staining.

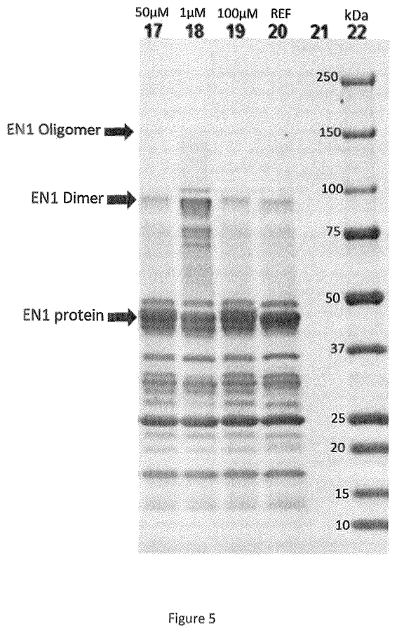

Lane 17 EN1 in 5 mM citrate/phosphate 250 mM Dextrose, 2.13 mM MgCl₂ pH 5, Glutathione 50 μM; Lane 18 EN1 in 5 mM citrate/phosphate 250 mM Dextrose, 2.13 mM MgCl₂ pH 5, Glutathione 1 μM; Lane 19 EN1 in 5 mM citrate/phosphate 250 mM Dextrose, 2.13 mM MgCl₂ pH 5, Glutathione 100 μM; Lane 21: blank; Lane 22: ladder.

### DETAILED DESCRIPTION

In the following, the invention will be explained in more detail with reference to the accompanying figures showing exemplified embodiments of the present invention. However, the present invention shall not to be limited in scope by the specific embodiments described herein. The following embodiments are given to enable those skilled in the art to more clearly understand and to practice the present invention.

Many of the approved protein pharmaceuticals, as well as those in clinical trials, are manufactured and stored as liquids, although historically more products were developed as solid formulations that were reconstituted to a liquid state just prior to injection. Proteins are inherently prone to form aggregates over time that may cause problems from the perspective of product quality, specifically product safety, efficacy, delivery or dosing, and marketability and are thus considered deleterious from the perspective of pharmaceutical product quality and performance (see for example Lundahl et al., 2021, RCS Chem. Biol., 2, 1004-1020). One major output of protein formulation development is the identification of inactivation, aggregation and/or degradation pathways of the respective protein. It is well known in the art that homeobox transcription factors such as EN protein are able to dimerize (Perez-Villamil et al, 2004, JBC 279, 38062-38071, Papadopoulos et al., 2012, Dev. Biol., 367, 78-89)) and the Inventor observed oligomerization of recombinant human EN protein during downstream production processing at room temperature leading to aggregation of the protein in aqueous solution (oligomerization/aggregation of the protein was detected by non-reduced SDS-Page electrophoresis assay).

In this context, the Inventors have elaborated a formulation that can inhibit or reduce the aggregation of EN protein in aqueous solution and developed a stabilized aqueous composition of Engrailed (EN) protein. Advantageously, the composition developed by the Inventors is compatible with intrathecal administration.

In an embodiment, the present invention provides stabilized aqueous composition of Engrailed (EN) protein comprising an effective amount or dose of an EN protein, a stabilizing concentration of reduced glutathione, an osmotic adjusting agent, and a buffering agent providing a pH less than 7, preferably less than 6, more preferably less than 5 and advantageously of about 4 to the formulation.

Unexpectedly, the Inventors have observed that aggregation of EN protein in aqueous composition can be inhibited or reduced in the presence of reduced glutathione.

In a preferred embodiment, the stabilized aqueous composition of Engrailed (EN) protein of the invention comprises reduced glutathione at a concentration from about 10 μM to about 100 μM. In an embodiment, the composition of the Invention contains about 50 μM of reduced glutathione. In a preferred embodiment, said reduced glutathione is reduced L-glutathione.

Also, the Inventors have surprisingly shown that the presence of osmotic adjusting agent in the EN protein aqueous composition improves EN protein stabilization, i.e. inhibits or reduces EN Protein aggregation.

In an embodiment, the stabilized aqueous composition of Engrailed (EN) protein has an osmolarity in a range from about 250 to about 350 mOsmol/L, more particularly in a range between about 270 mOsmol/L and about 320 mOsmol/L. Examples of suitable osmotic adjusting agents include, but are not limited to, sugars such as glucose (e.g., dextrose), poly(glucose), fructose, dextrans, glycerol, sorbitol, mannitol, trehalose, mannose, polyanions, and the like and combinations thereof. Other osmotic adjusting agents, which may be non-sugar agent and that function as an equivalent, could be a viable substitute, such as small amino acids.

In a preferred embodiment, the stabilized aqueous composition of Engrailed (EN) protein is for an intrathecal administration and the osmotic adjusting agent is preferably dextrose. In a preferred embodiment, the osmotic adjusting agent is dextrose. In an embodiment, the stabilized aqueous composition of Engrailed (EN) protein of the invention comprises dextrose at a concentration from about 220 to about 320 mM, advantageously from about 240 mM to about 290 mM. In a preferred embodiment, it is about 250 mM.

Advantageously, it was also found by the inventors that the inclusion of magnesium salt substantially reduces the aggregation of EN compositions. Accordingly, in one embodiment, the stabilized aqueous composition of Engrailed (EN) protein of the invention comprises a magnesium salt, preferably MgCl₂. In a preferred embodiment, magnesium salt is present at a concentration from about 0.1 mM to about 3 mM. In a preferred embodiment, the stabilized aqueous composition of Engrailed (EN) protein of the invention comprises about 2.13 mM MgCl₂.

Advantageously, the EN protein aqueous composition of the Invention comprises dextrose combined with magnesium chloride.

Advantageously, it was also found by the Inventors that the inclusion of low level (i.e. less than 0.2%) of nonionic surfactant substantially reduces adherence to support and helps to stabilize EN protein. Accordingly, in one embodiment, the stabilized aqueous composition of Engrailed (EN) protein of the invention comprises a stabilizing concentration of a nonionic detergent. Nonionic surfactants that may be used in the compositions of the present invention are known in the art, and include, without limitation, Polysorbate 80 (Tween 80), Polysorbate 20 (Tween 20), and various poloxamers or pluronics, including Pluronic F-68, or mixtures thereof. In a preferred embodiment, the nonionic surfactant is Polysorbate 20. In certain embodiments, the surfactant is present at a concentration from about 0% to about 0.2%. In a preferred embodiment, the surfactant is at a concentration from about 0% to about 0.01%. In another preferred embodiment, the surfactant is at a concentration of about 0.005%.

In an embodiment, the present invention provides a stabilized aqueous composition of Engrailed (EN) protein comprising:
effective amount or dose of an EN protein;
from about 10 μM to about 100 μM reduced glutathione;
from about 240 to about 290 mM of an osmotic adjusting agent, preferably dextrose;
from about 0.1 mM to about 3 mM of MgCl₂;
from 0 to 0.01% polysorbate 20; and
a buffering agent for maintaining pH at about 4.

In an embodiment, the present invention provides a stabilized aqueous composition of Engrailed (EN) protein comprising an effective amount or dose of EN protein of from about 0.05 mg/ml and about 1 mg/ml. In some embodiments, EN is present in a composition provided herein at a concentration from about 0.05 mg/ml and about 0.9 mg/mL. In some embodiments, EN is present at a concentration from about 0.1 mg/ml and about 0.9 mg/mL. In some embodiments, EN may be present at about 0.6 mg/ml.

In an embodiment, the buffering agent is sodium acetate/acetic acid.

In an advantageous aspect, the present invention provides a stabilized aqueous composition of Engrailed (EN) protein comprising:
about 0.6 mg/ml of EN protein;
about 250 mM of dextrose;
about 2.13 mM of MgCl₂;
about 50 μM of reduced glutathione;
about 0.005% polysorbate 20; and
about 5 mM sodium acetate/acetic acid for maintaining a pH about 4.

As used herein, the term “EN” refers to an Engrailed protein. In the context of the present invention, an EN protein embraces any EN protein, for example, Engrailed 1 (EN1) or Engrailed 2 (EN2), as well as mixture thereof, from a mammal such as a primate, human, monkey, rabbit, pig, bovine, or rodent, preferably human, and biologically active derivatives thereof. Mutant and variant EN proteins having activity are also embraced, as are functional fragments and fusion proteins of the EN protein. Exemplary EN1 protein or polypeptide includes, without limitation, human EN1 protein or polypeptide having primary amino acid sequence as annotated under Genbank accession number AAA 53502.2 or NCBI NP_001417.3. Exemplary EN2 protein or polypeptide includes, without limitation, human EN2 protein or polypeptide having primary amino acid sequence as annotated under Genbank accession number AAA 53504.2 or NCBI NP_001418.2. In certain embodiments of the formulations provided herein, the EN protein is a human EN or recombinant human EN protein, or a biologically active derivative or fragment thereof. In one embodiment, the EN protein is a human EN1.

As used herein, the term “biologically active derivative” refers to any polypeptide with substantially the same biological function as EN. The polypeptide sequences of the biologically active derivatives may comprise deletions, additions and/or substitution of one or more amino acids whose absence, presence and/or substitution, respectively, do not have any substantial negative impact on the biological activity of polypeptide.

As used herein, the terms “EN” and “biologically active derivative”, respectively, also include polypeptides obtained via recombinant DNA technology or synthetically. The recombinant EN, e.g. recombinant human EN may be produced by any method known in the art. This includes any method known in the art for (i) the production of recombinant DNA by genetic engineering, e.g. via reverse transcription of RNA and/or amplification of DNA, (ii) introducing recombinant DNA into prokaryotic or eukaryotic cells by transfection, i.e. via electroporation or microinjection, (iii) cultivating said transformed cells, e.g. in a continuous or batchwise manner, (iv) expressing EN, e.g. constitutively or upon induction, and (v) isolating said EN, e.g. from the culture medium or by harvesting the transformed cells, in order to (vi) obtain substantially purified recombinant EN, e.g. via anion exchange chromatography or affinity chromatography.

As used herein, an “effective amount or dose” or “sufficient amount or dose” refers to a quantity of a compound that produces effects for which it is administered. The exact quantity will depend on the purpose of the treatment and will be ascertainable by one skilled in the art using known techniques.

As used herein, the term “about” denotes an approximate range of plus or minus 10% from a specified value. For instance, the language “about 20%” encompasses a range of 18-22%. As used herein, about also includes the exact amount. Hence “about 20%” means “about 20%” and also “20%.”

As used herein, the expression “aqueous composition” refers to a composition comprising water as solvent.

As used herein, “storage” means that a composition of the invention is not immediately administered to a subject once prepared, but is kept for a period of time under particular conditions (e.g., particular temperature, etc.) prior to use. For example, a liquid or lyophilized composition can be kept for days, weeks, months or years, prior to administration to a subject under varied temperatures such as frozen (=<−65° C. or −15° C. to −25° C.) refrigerated (0° to 10° C.) or room temperature (e.g., temperature up to 32° C.).

According to special embodiments, the compositions of the invention reduce or delay dimerization, oligomerization, and/or aggregation of an EN protein over time. In one embodiment, the stabilized aqueous composition of Engrailed (EN) protein of the invention are stable when stored at temperatures up to at least about −65° C. for at least about 6 months. In other embodiments, the compositions provided herein retain significant EN activity when stored for extended periods of time.

As used herein, “stabilized composition of a protein” or “stable protein in the composition” means that the EN protein has maintained the same oligomerization state, preferably has remained as a monomer, and/or does not form aggregate in the said composition. A person of ordinary skill in the art will know how to determine oligomerization/aggregation state of a protein, for instance by non-reduced SDS-Page electrophoresis or size exclusion chromatography (SEC). In a preferred embodiment, “stabilized composition of EN protein” means that the EN protein dimers, oligomers and/or aggregation in said composition is less than about 5%, preferably less than about 3% of the total EN protein amount.

In a related aspect, the present invention provides a stabilized lyophilized composition of EN protein, wherein the formulation is lyophilized from the stabilized aqueous composition of Engrailed (EN) protein as provided herein. Lyophilization can be performed according to method of the art.

Generally, the stabilized aqueous composition of Engrailed (EN) protein provided herein are suitable for pharmaceutical administration.

In a preferred embodiment, the EN composition provided herein is sterile and comprises low endotoxin levels (according to European Pharmacopeia).

In some embodiments, the EN composition provided herein may further comprise one or more pharmaceutically acceptable excipients, carriers, and/or diluents. In addition, the composition provided herein may further comprise other medicinal agents, carriers, adjuvants, diluents, tissue permeation enhancers, solubilizers, and the like. Methods for preparing compositions and formulations for pharmaceutical administration are known to those skilled in the art.

The stabilized aqueous composition of Engrailed (EN) protein provided herein may be formulated for administration via known methods, as a bolus or by continuous infusion over a period of time. It is particularly adapted for administration by intrathecal or intracerebroventricular routes.

Stability of the aqueous composition of Engrailed (EN) protein may be measured by one or more biophysical properties of the EN protein in the formulation. Non-limiting examples of properties that may be used to assess stability include the degree of mono- or poly-dispersity of the protein, the extent of dimerization, oligomerization, or aggregation of the EN protein. One skilled in the art will readily know of other measures of stability that may be employed to assess the stability of an EN formulation, including without limitation, size exclusion chromatography (SEC), dynamic or static light scattering, RP-HPLC, ion-exchange chromatography, polyacrylamide gel electrophoresis, non-reduced SDS-Page electrophoresis and the like.

In certain embodiments, the aqueous composition of Engrailed (EN) protein of the invention may be stable for an extended period of time when stored at a certain temperature, for example, at about −65° C., −20° C., 4° C., 18° C., room temperature, 25° C., 30° C., 35° C., 37° C., 40° C., or higher. In some embodiments, an extended period of time is at least about a week. In other embodiments, an extended period of time may comprise at least about 2 weeks, or at least about 3 week, or at least about 1 month, or at least about 2 months, or at least about 3, 4, 5, 6, 7, 8, 9, 10, 11, 12, 14, 16, or 18 months. In yet other embodiments, the composition of the invention may be stable for at least about 2, 3, 4, 5 or more years.

In an embodiment, the invention relates to stabilized aqueous composition of Engrailed (EN) protein substantially free of aggregated EN protein, dimeric EN protein, oligomeric EN protein or mixture thereof. In a preferred embodiment, the stabilized aqueous composition of Engrailed (EN) protein is substantially free of aggregated EN protein, dimeric EN protein, oligomeric EN protein or mixture thereof when it contains less than 5%, preferably less than 3%, of aggregated EN protein, dimeric EN protein, oligomeric EN protein or mixture thereof, or less than 2%, or less than 1%.

In another embodiment of the invention, a stabilized aqueous composition of Engrailed (EN) protein of the Invention has an EN protein population consisting of at least about 95% EN protein monomers after storage for an extended period of time. In other embodiments, the EN protein composition has at least about 97% EN protein monomers, or at least about 98%, 99%, or a higher percentage of EN protein monomers.

In some embodiments, an EN protein used in the formulations provided herein may be expressed, produced, or purified according to a method well known in the art.

Recombinant EN proteins can be produced by expression in any suitable prokaryotic or eukaryotic host system. In one embodiment, the EN proteins can be expressed in bacterial cells, yeast cells, insect cells, avian cells, mammalian cells, and the like. Examples of eukaryotic cells include, without limitation, mammalian cells, such as CHO, COS, HEK 293, BHK, SK-Hep-1, and HepG2; insect cells, for example SF9 cells, SF21 cells, S2 cells, and High Five cells; and yeast cells, for example Saccharomyces, Pichia or Schizosaccharomyces cells.

In one embodiment, the cells may be any bacterial cell that can be cultured, preferably in a manufacturing process (i.e., at least 1 liter), to produce a desired EN protein. In a preferred embodiment, the cell line is a E. coli cell line.

A wide variety of vectors can be used for the expression of an EN protein (e.g. EN1) and can be selected from eukaryotic and prokaryotic expression vectors. In certain embodiments, a plasmid vector is contemplated for use in expressing an EN protein (e.g. EN1). The plasmid will comprise a nucleotide sequence encoding an EN protein (e.g. EN1) operable linked to one or more control sequences, such as promoter, for example inducible promoter. According to certain embodiments, a viral vector is used to introduce a nucleotide sequence encoding an EN protein (e.g. EN1) into a host cell for expression. The viral vector will comprise a nucleotide sequence encoding an EN protein (e.g. EN1) operable linked to one or more control sequences, for example, a promoter. Non-limiting examples of virus vectors that may be used to deliver a nucleic acid include Adenoviral vectors, AAV vectors, and Retroviral vectors. Non-limiting examples of vectors for prokaryotic expression include plasmids such as pRSET, pET, pBAD, etc., wherein the promoters used in prokaryotic expression vectors include lac, trc, trp, recA, araBAD, etc. Examples of vectors for eukaryotic expression include: (i) for expression in yeast, vectors such as pAO, pPIC, pYES, pMET, using promoters such as AOXI, GAP, GALI, AUG I, etc; (ii) for expression in insect cells, vectors such as pMT, pAc5, plB, pMIB, pBAC, etc., using promoters such as PH, pl O, MT, Ac5, OplE2, gp64, polh, etc., and (iii) for expression in mammalian cells, vectors such as pSVL, pCMV, pRc/RSV, pcDNA3, pBPV, etc., and vectors derived form viral systems such as vaccinia virus, adeno-associated viruses, herpes viruses, retroviruses, etc., using promoters such as CMV, SV40, EF-1, UbC, RSV, ADV, BPV, and β-actin.

In certain embodiments, EN expression can comprise the use of a cell culture system operated under a batch or continuous mode of operation. For example, when batch cell cultures are utilized, they may be operated under single batch, fed-batch, or repeated-batch mode. Likewise, continuous cell cultures may be operated under, for example, perfusion, turbidostat or chemostat mode. Batch and continuous cell cultivation may be performed under either suspension or adherence conditions. When operated under suspension conditions, the cells will be freely suspended and mixed within the culture medium. Alternatively, under adherence conditions, the cells will be bound to a solid phase, for example, a microcarrier, a porous microcarrier, disk carrier, ceramic cartridge, hollow fiber, flat sheet, gel matrix, and the like.

A batch culture is typically a large scale cell culture in which a cell inoculum is cultured to a maximum density in a tank or fermenter, and harvested and processed as a single batch. A fed-batch culture it typically a batch culture which is supplied with either fresh nutrients (e.g., growth-limiting substrates) or additives (e.g., precursors to products) to maintain cell growth and reach higher biomass. The feed solution is usually highly concentrated to avoid dilution of the bioreactor. In a repeated-batch culture, the cells are placed in a culture medium and grown to a desired cell density. To avoid the onset of a decline phase and cell death, the culture is then diluted with complete growth medium before the cells reach their maximum concentration. The amount and frequency of dilution varies widely and depends on the growth characteristics of the cell line and convenience of the culture process. The process can be repeated as many times as required and, unless cells and medium are discarded at subculture, the volume of culture will increase stepwise as each dilution is made. The increasing volume may be handled by having a reactor of sufficient size to allow dilutions within the vessel or by dividing the diluted culture into several vessels. The rationale of this type of culture is to maintain the cells in an exponentially growing state. In certain embodiments, an EN protein may be recovered after harvesting the supernatant of a batch culture.

Methods for the expression and purification of recombinant EN protein are also known in the art.

Additionally, another aspect of the invention relates to methods for the purification of recombinant EN protein. In one embodiment, recombinant EN protein is expressed in recombinant bacteria and purified from the resulting conditioned media by a series of chromatography and filtration/ultrafiltration steps.

In one embodiment, a method is provided for providing a EN protein (e.g. EN1) composition, the method comprising the steps of (a) culturing a cell harboring a nucleic acid encoding a EN protein (e.g. EN1) in a culture medium; (b) performing a lysis step to release the EN protein in the supernatant n; (c) clarifying the supernatant containing the EN protein (d) purifying the EN protein by a series of chromatography and filtrations/ultrafiltration steps; and e) formulating the EN protein (e.g. EN1) composition according to a formulation provided herein, thereby providing a EN protein (e.g. EN1) formulation.

In one embodiment, the present invention provides a method for manufacturing a stabilized aqueous composition of Engrailed (EN) protein, the method comprising the steps of: (i) expressing an EN protein (e.g. EN1), or a biologically active derivative thereof, by culturing a cell harboring a nucleic acid encoding a EN protein (e.g. EN1) in a cell cultured in a medium; (ii) performing a lysis step to release the EN protein in the supernatant; (iii) clarifying the supernatant containing the EN protein; (iv) purifying the EN protein (e.g. EN1); and (v) preparing a composition comprising an effective amount or dose of an EN protein, an osmotic adjusting agent, a stabilizing concentration of reduced glutathione and a buffering agent providing a pH less than 7, preferably less than 6, more preferably less than 5 and advantageously of about 4 to the formulation.

In one embodiment, the present invention provides a method for manufacturing a stabilized aqueous composition of Engrailed (EN) protein, the method comprising the steps of: (i) expressing an EN protein (e.g. EN1), or a biologically active derivative thereof, by culturing a cell harboring a nucleic acid encoding a EN protein (e.g. EN1) in a cell cultured in a medium; (ii) performing a lysis step to release the EN protein in the supernatant; (iii) clarifying the supernatant containing the EN protein; (iv) purifying the EN protein (e.g. EN1); and (v) preparing a composition comprising (a) about 0.6 mg/ml EN protein; (b) about 250 mM dextrose; (c) about 50 μM reduced glutathione; (d) about 2.13 mM of MgCl₂; (e) 0.005% polysorbate 20; and (f) a buffering agent for maintaining a pH less than 7, preferably less than 6, more preferably less than 5 and advantageously of about 4.

### Administration and Methods of Treatment

The compositions of the Invention can be administered for therapeutic or prophylactic treatments. Generally, for therapeutic applications, formulations are administered in a therapeutically effective dose to a patient with a disease or condition associated with death of neurons, preferably death of motor neurons, preferably Amyotrophic lateral sclerosis (ALS).

Formulations and amounts effective for these uses will depend upon the severity of the disease or condition and the general state of the patient's health. Single or multiple administrations of the formulations may be administered depending on the dosage and frequency as required and tolerated by the patient.

A “patient” or “subject” for the purposes of the present invention includes both humans and other animals, particularly mammals. The compositions, formulations, and methods are applicable to both human therapy and veterinary applications. In a particular embodiment, the patient is a mammal, preferably a human. Other known treatments and therapies for conditions associated with death of neurons, preferably death of motor neurons, can be used in combination with the formulations and methods provided by the invention.

The preferred delivery route is intrathecal or intracerebroventricular administration, more preferred delivery route is intrathecal. The volume of intrathecal injections ranges from 0.1 to 20 mL.

Doses ranging from 0.01 mg to 18 mg of EN protein may be administered as intrathecal or intracerebral administrations, more particularly intrathecal administration to a patient with a disease or condition associated with death of neurons, preferably death of motor neurons, preferably Amyotrophic lateral sclerosis (ALS).

According to special embodiment, the stabilized aqueous composition of Engrailed (EN) protein can be administered monthly or bimonthly.

According to one special embodiment, the stabilized aqueous composition of Engrailed (EN) protein (preferably EN1 protein) of the invention is administered to a patient with a disease or condition associated with death of neurons, preferably death of motor neurons, preferably Amyotrophic lateral sclerosis (ALS). According to particular embodiment, administration of the stabilized aqueous composition of Engrailed (EN) protein (preferably EN1 protein) of the invention to a patient with a disease or condition associated with death of neurons, preferably death of motor neurons, preferably Amyotrophic lateral sclerosis (ALS), results in controlling motoneurons death progress, preferably in stopping motoneurons death.

In another aspect of the invention, kits are provided for the treatment of a disease or condition associated with death of neurons, preferably death of motor neurons, more preferably Amyotrophic lateral sclerosis (ALS). In one embodiment, the kit comprises a composition of EN protein, as provided above. In some embodiments, the kits provided herein may contain one or more dose of a liquid or lyophilized composition as provided herein. According to special embodiment, e.g. kits with lyophilized EN protein composition, the kits further contains a suitable liquid for reconstitution of the liquid formulation, for example, artificial CSF or a pharmaceutically acceptable buffer. In some embodiments, the kits may comprise an EN protein composition prepackaged in a syringe for administration by a health care professional or for home use.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

Buffer exchange steps have been identified as promoting oligomerization of EN protein during downstream protein production process.

Buffer exchange small scale models (dialysis and ultrafiltration centrifuge tubes) have been selected and used in order to screen different formulations of EN protein and EN protein oligomerization level.

Two main analytical methods have been used to measure EN protein oligomerization: Size Exclusion Chromatography (SEC) HPLC and non-reduced electrophoresis SDS-Page with Coomassie blue staining.

### Example 1: Acidic pH Effect

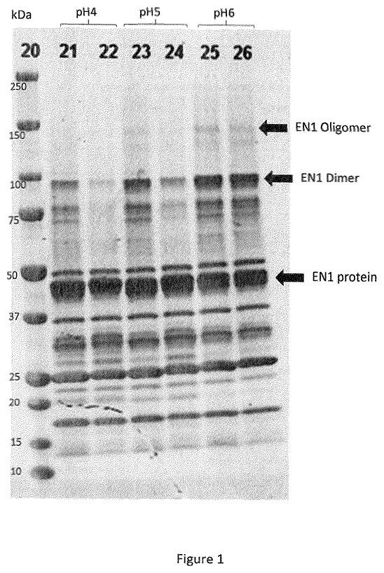

Acidic pH effect on EN1 protein oligomerization has been analysed with different compositions of EN1 protein in two different test models (dialysis or small scale ultrafiltration) (FIG. 1):
EN1 protein composition comprising 5 mM citrate/phosphate, 250 mM Dextrose, 2.13 mM MgCl₂, pH 4 after dialysis;
EN1 protein composition comprising 5 mM citrate/phosphate, 250 mM Dextrose, 2.13 mM MgCl₂, pH 4 after small scale ultrafiltration;
EN1 protein composition comprising 5 mM citrate/phosphate, 250 mM Dextrose, 2.13 mM MgCl₂, pH 5 after dialysis;
EN1 protein composition comprising 5 mM citrate/phosphate, 250 mM Dextrose, 2.13 mM MgCl₂, pH 5 after small scale ultrafiltration;
EN1 protein composition comprising 5 mM citrate/phosphate, 250 mM Dextrose, 2.13 mM MgCl₂, pH 6 after dialysis;
EN1 protein composition comprising 5 mM citrate/phosphate, 250 mM Dextrose, 2.13 mM MgCl₂ pH 6 after small scale ultrafiltration,

EN protein oligomerization has been determined using non-reduced SDS-Page electrophoresis method. FIG. 1 shows that composition with pH 4 results in limiting oligomerization of EN1 protein compared to pH 5 and 6.

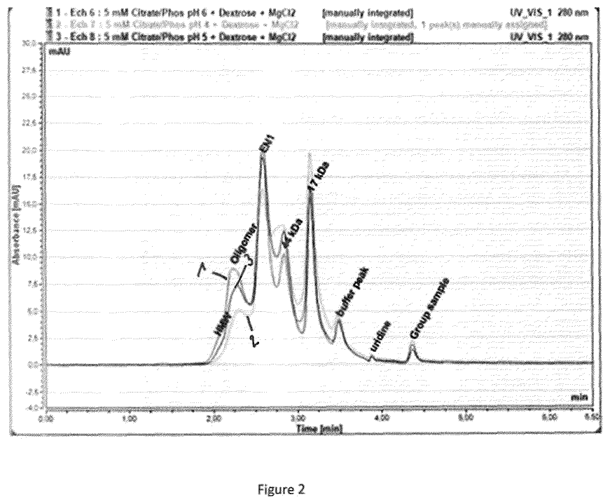

Similarly, EN protein oligomerization in compositions with dialysis (see above) has been determined using SEC-HPLC method with high molecular weight (HMW) detected at 280 nm (FIG. 2). Results confirm that composition with pH 4 has the lower oligomer peaks and prevents EN1 protein oligomerization more efficiently than compositions with pH 5 and 6.

### Example 2: Glutathione Effect

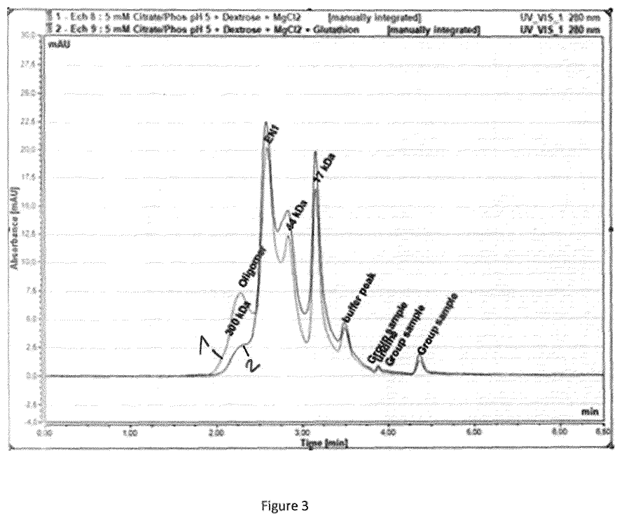

EN1 protein oligomerization was measured by SEC-HPLC, in composition comprising EN1 protein, 5 mM Citrate/phosphate, 250 mM Dextrose, 2.13 mM MgCl₂, pH 5 in absence or in presence of 50 μM reduced Glutathione (FIG. 3).

FIG. 3 shows that the amount of EN1 oligomer is dramatically reduced in the presence of Glutathione in the EN1 protein composition.

### Example 3: Glutathione Concentration Effect

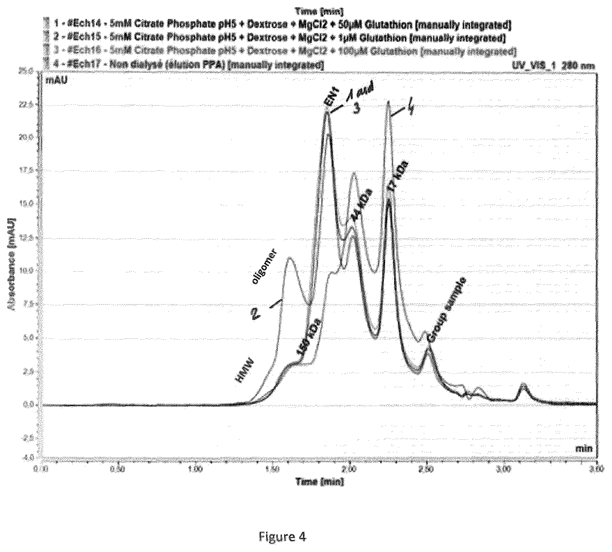

EN1 protein oligomerization was measured by SEC-HPLC, in composition comprising EN1 protein, 5 mM Citrate/phosphate, 250 mM Dextrose, 2.13 mM MgCl₂, pH 5 and various concentration of reduced Glutathione (1 μM, 50 μM or 100 μM) (FIG. 4).

FIG. 4 illustrates that presence of Glutathione 50 μM or 100 μM in composition prevents EN1 protein oligomerization.

Similar analysis and conclusion have been observed using non-reduced SDS-Page electrophoresis method.

1. A stabilized aqueous composition of Engrailed (EN) protein comprising an effective amount or dose of an EN protein, a stabilizing concentration of reduced glutathione, an osmotic adjusting agent, and a buffering agent providing a pH less than 7.

2. The composition of claim 1, wherein said stabilizing concentration of reduced glutathione is selected from about 10 μM to about 100 μM.

3. The composition of claim 1, wherein its osmolarity ranges from about 250 to about 350 mOsmol/L.

4. The composition of claim 1, wherein the osmotic adjusting agent concentration is from about 220 to about 320 mM.

5. The composition of claim 1, wherein the osmotic adjusting agent is dextrose.

6. The composition of claim 1, wherein the composition comprises magnesium salt.

7. The composition of claim 6, wherein the magnesium salt concentration is from about 0.1 mM to about 3 mM.

8. The composition of claim 1, wherein the composition comprises nonionic detergent.

9. The composition of claim 8, wherein the nonionic surfactant is Polysorbate 20.

10. The composition of claim 8, wherein the nonionic surfactant concentration is from about 0% to about 0.2%.

11. The composition of claim 1, wherein the composition comprises:
effective amount or dose of an EN protein;
from about 10 μM to about 100 μM reduced glutathione;
from about 240 to about 290 mM of an osmotic adjusting agent;
from about 0.1 mM to about 3 mM of MgCl2;
from 0 to 0.01% polysorbate 20; and
a buffering agent for maintaining pH at about 4.

12. The composition of claim 11, wherein the effective amount or dose of EN protein is from about 0.05 mg/mL and about 1 mg/mL.

13. The composition of claim 2, wherein its osmolarity ranges from about 250 to about 350 mOsmol/L.

14. The composition of claim 2, wherein the osmotic adjusting agent concentration is from about 220 to about 320 mM.

15. The composition of claim 2, wherein the osmotic adjusting agent is dextrose.

16. The composition of claim 2, wherein the composition comprises magnesium salt.

17. The composition of claim 2, wherein the composition comprises nonionic detergent.

18. The composition of claim 9, wherein the nonionic surfactant concentration is from about 0% to about 0.2%.

19. The composition of claim 2, wherein the composition comprises:
effective amount or dose of an EN protein;
from about 10 μM to about 100 μM reduced glutathione;
from about 240 to about 290 mM of an osmotic adjusting agent;
from about 0.1 mM to about 3 mM of MgCl2;
from 0 to 0.01% polysorbate 20; and
a buffering agent for maintaining pH at about 4.
